(19) United States
(12) Patent Application Publication
(10) Pub. No.: US 2006/0089582 A1
(43) Pub. Date: Apr. 27, 2006

(54) Back protection system

(76) Inventor: Vladimir Rakhmanin, Rockville, MD (US)

Correspondence Address: LOWE HAUPTMAN GILMAN AND BERNER, LLP, 1700 DIAGONAL ROAD, SUITE 300 /310, ALEXANDRIA, VA, 22314, US

(21) Appl. No.: 11/254,808

(22) Filed: Oct. 21, 2005

Related U.S. Application Data
(60) Provisional application No. 60/620,438, filed on Oct. 21, 2004.

(51) Int. Cl. A61F 5/00

(52) U.S. Cl. 602/19; 602/2

(57) ABSTRACT

A back protection system is described. The back protection system includes a belt for placement on a user body, a support pad operatively positioned at a central region of the belt, a heating element positioned adjacent a side of the support pad and operatively connected with the belt, and a power source connected to the heating element and arranged to cause the heating element to generate heat adjacent said support pad to a user's body.

### FIELD OF THE INVENTION

The present embodiments relate to a back protection system.

### BACKGROUND

It is known in the art to use a support belt to prevent back pain.

Low back pain is a common musculoskeletal symptom that may be either acute or chronic. Caused by a variety of diseases and disorders that affect the lumbar spine, lower back pain is often accompanied by sciatica, which is pain that involves the sciatic nerve and is felt in the lower back, the buttocks, the back, and sides of the thighs, and possibly the calves. A symptom that affects 80% of the general United States population at some point in life with sufficient severity to cause absence from work, lower back pain is a common reason for visits to primary care doctors, and is estimated to cost the American economy billions of dollars every year.

As many sufferers of lower back pain can readily attest, efforts to remain physically active can be fraught with challenges. Whether one desires to frequent a gym to retain muscle tone or to simply jog in the mornings for a cardiovascular workout, frequent back spasms can render exercise regimens extremely difficult and sometimes impossible. Even the most common, everyday tasks can be frustrating to complete. The movements required to dress, bathe, or simply walk through the house can sometimes result in episodes of sharp, stabbing aches. Even sitting for long periods of time can be extremely uncomfortable for those afflicted with lower back pain. Driving or riding in the car, sitting behind a desk at work, or trying to relax at home is often punctuated with painful back stress.

### SUMMARY

The present embodiments provide a back protection system. The back protection system includes a belt for placement on a user body, a support pad operatively positioned at a central region of the belt, a heating element positioned adjacent a side of the support pad and operatively connected with the belt, and a power source connected to the heating element and arranged to cause the heating element to generate heat adjacent said support pad to a user.

A method embodiment of applying heat to a user's back region using a back protection system having a support pad at a central region and at least one heating element adjacent a side of the support pad includes positioning the back protection system about a user's body and aligning the support pad with the user's spine. The heating element is activated to generate heat to the user's body.

There are numerous advantages to the below-described back protection system.

An embodiment of the present invention is a specially designed, lumbar support system with heat capabilities. One embodiment of the present application provides relief to suffers of lower back pain, and prevents the onset of such pain in non-sufferers, while exercising, walking, or simply sitting or reclining. Further, in one embodiment, power source requirements are reduced as heat is generated toward effected back muscles and not toward the spine. To this end, a support pad aids in positioning the back protection system in order to direct heat to the muscles which need to be heated.

Still other advantages of embodiments according to the present invention will become readily apparent to those skilled in the art from the following detailed description, wherein the preferred embodiments of the invention are shown and described, simply by way of illustration of the best mode contemplated of carrying out the invention. As will be realized, the invention is capable of other and different embodiments, and its several details are capable of modifications in various obvious respects, all without departing from the invention.

### DESCRIPTION OF THE DRAWINGS

The present invention is illustrated by way of example, and not by limitation, in the figures of the accompanying drawings, wherein elements having the same reference numeral designations represent like elements throughout and wherein:

FIG. 1 is a frontal view of an embodiment;

FIG. 2 is a frontal view of another embodiment;

FIG. 3 is a frontal view of another embodiment;

FIG. 4 is a frontal view of another version of the FIG. 3 embodiment; and

FIG. 5 is a rear view of the FIG. 1 embodiment positioned on a wearer's body.

### DETAILED DESCRIPTION

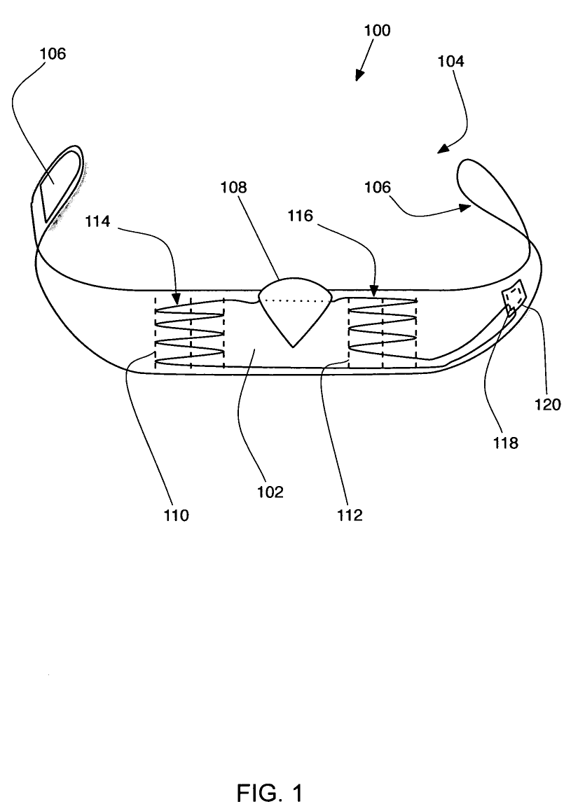

Recognizing the need for an ergonomic device that offers optimal lower back support while exercising or simply sitting, The present inventor has conceived of a novel device designated the Back Protection System (BPS). Simply stated and depicted in an embodiment in FIG. 1, BPS 100 is a wearable belt specially designed to provide heated lumbar support for the lower back of a wearer. The belt is fabricated of a durable yet pliable material such as elastic or nylon and has an interior face 102 and an exterior face 104. Interior face 102 is closest to a wearer's body during use.

The belt itself features a fastening system 106, such as strips of a hook and loop fastener system commonly known as VELCRO to facilitate securing BPS 100 about the wearer. In an embodiment, fastening system 106 includes a hook patch affixed to exterior face 104 at one end of BPS 100 and a corresponding loop patch affixed to interior face 102 at an opposite end of the BPS. In alternate embodiments, BPS 100 includes different forms of fastening system 106, e.g., notched belt and buckle, etc.

In an embodiment, BPS 100 measures between approximately twenty-four and forty-two inches (24″-42″) in adjustable length. BPS 100, according to an embodiment, features widths of approximately five inches (5″) in the front at either end of the BPS and approximately seven to eight inches (7″-8″) at its widest point in the back toward the center of the length of the BPS, i.e., at the point of the BPS located adjacent a wearer's spine. Positioned toward the middle of interior face 102, a lumbar support pad 108, e.g., an approximately diamond-shaped pad as depicted in FIG. 1, constructed of soft, rubberized foam is attached to BPS 100. In an embodiment, support pad 108 is attached by hook and loop fasteners. In a further embodiment, support pad 108 is integrally attached to BPS 100 via stitching. In another embodiment, support pad 108 is directly fastened within or to interior face 102 of BPS 100 via a pad fastener, e.g., stitching, glue, hook and loop fastener, etc. A purpose of lumbar support pad 108 is to center BPS 100 on the back of the wearer such that heating elements (114, 116 described in detail below) are positioned over muscles which need to be heated.

A series of optional horizontal, reinforcement strips 110, 112 are positioned on a respective left and right side of support pad 108. In an embodiment, reinforcement strips 110, 112 are made of a plastic material and provide additional support to the wearer's back while minimizing bunching or gathering of BPS 100. Optional reinforcement strips 110, 112 are depicted via dashed lines in FIG. 1.

Electrically connected behind lumbar support pad 108 and internally contained within the material of the belt are several lengths of heating elements 114, 116, such as coils, wires, etc., powered by a power source 118, such as a standard 9 volt, AA, C alkaline or other battery or other rechargeable and non-rechargeable power sources. According to an embodiment, power source 118 resides in a battery pocket 120 affixed to or formed as a part of BPS 100. In one particular embodiment, a power plug may replace power source 118 or, in a further embodiment, be used in addition to the power source in order to provide power to BPS 100 and/or charge the power source.

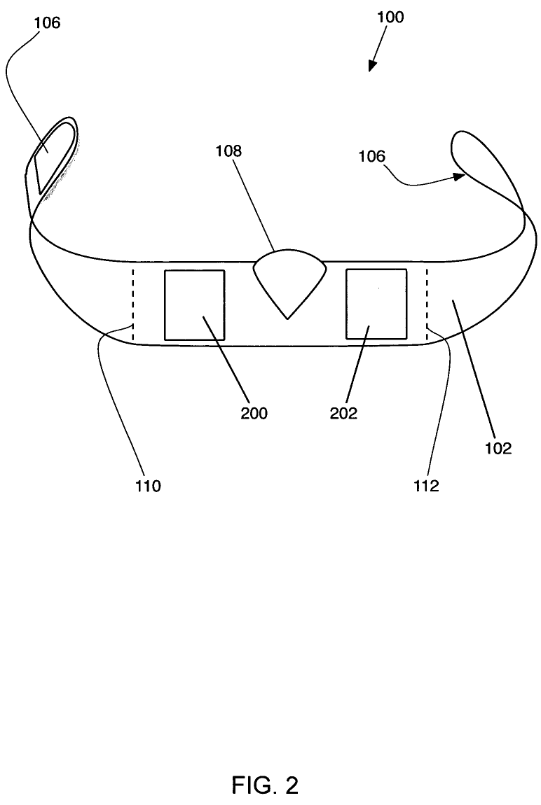

In another embodiment, described in further detail below with reference to FIG. 2, power source 118 and heating elements 114, 116 may be replaced with one or more self-contained heating elements, such as a chemical or electrical-based heating unit, e.g., a THERMACARE pad, adjacent interior face 102 of BPS 100. In a further embodiment, heating elements 114, 116 are separately connected to individual power sources (not shown) positioned on corresponding sides of support pad 108. In a further embodiment, the heat generated by heating elements 114, 116 is individually or jointly controllable by a controller (not shown), e.g., an on/off switch, a high/low/off switch, a rheostat, and other incrementally or variable adjustable control device, etc.

Positioning heating elements 114, 116 over only muscles which are to be heated reduces the amount of energy consumed by BPS 100 in heated use and conversely increases the amount of time BPS 100 may be used to provide heat to the back of the wearer. That is, the energy savings obtained by selectively applying heat to the user's back translates to extended heat-generating time of heating elements 114, 116 in particular embodiments, e.g., electrical-based heating elements. In another embodiment, a single heating element 114 or 116 may be affixed to BPS 100 and positioned on either the left or right side of support pad 108 on interior face 102. In a further embodiment, single heating element 114, 116 may be affixed to exterior face 104; however, the efficiency of transferring the heat to a wearer's back will be greatly decreased.

In an embodiment and as depicted in FIG. 1, heating elements 114, 116 are positioned on either side of lumbar support pad 108. Application of electric current to heating element 114, 116 causes the element to generate heat and due to placement of BPS 100 on a wearer's back, the generated heat transfers toward back muscles of the wearer. In a first embodiment, the amount of heat generated by heating elements 114, 116 is preset and unchangeable by the wearer. The wearer may activate or deactivate one or the other or both of heating elements 114, 116 as needed either by using a switch (not shown) or by otherwise disconnecting power source 118. In a second embodiment, a variable control (not shown) is useable to control the amount of heat generated by heating elements 114, 116.

In the FIG. 1 embodiment, heating elements 114, 116 on either side of lumbar support pad 108 are electrically connected together and form a single heating system. In alternate embodiments, separate heating elements may be used electrically connected to either a single power source or separate power sources.

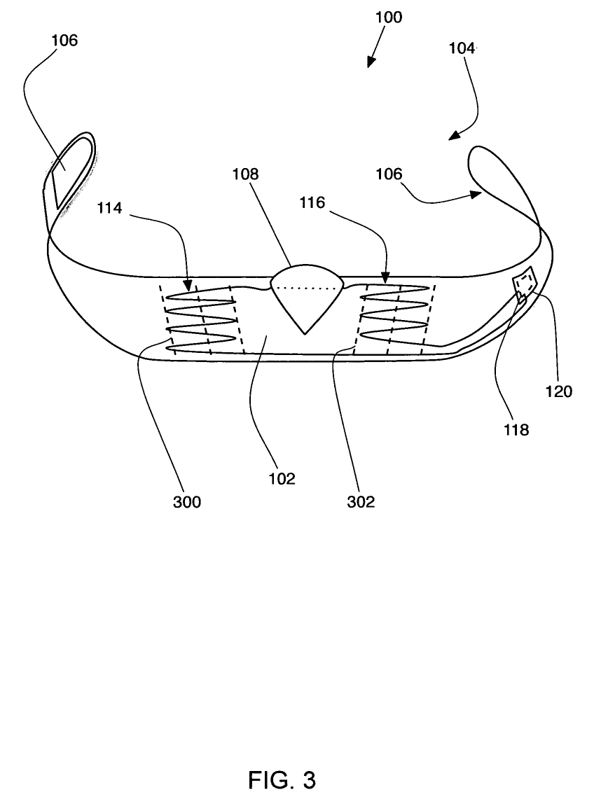

In a further embodiment, optional set of reinforcement strips 110, 112, e.g., stays, are positioned on or in the belt. In an embodiment, the strips 110, 112 are affixed to the exterior of BPS 100 and in another embodiment, the strips are woven into the material of the BPS. Strips 110, 112 are vertically aligned on either side of lumbar support pad 108. FIG. 3 depicts an embodiment of BPS 100 in which strips 300, 302 (corresponding to strips 110, 112 of FIG. 1) are obliquely aligned with the spine of a wearer when BPS 100 is positioned on the wearer. Strips 110, 112 provide support to the wearer while allowing side-to-side bending flexibility of the wearer. In a further particular embodiment, strips 110, 112 are made of plastic. The number of strips in BPS 100 varies based on the size of the belt and may range from a pair (one strip disposed on either side of support pad 108) to a plurality of strips.

In a still further embodiment, the material of BPS 100 between heating element 110 and the proximal end of the BPS and between heating element 112 and the proximal end of the BPS is a stretchable material. In this manner, BPS 100 is able to flex to a particular degree in order to provide additional flexibility to the wearer.

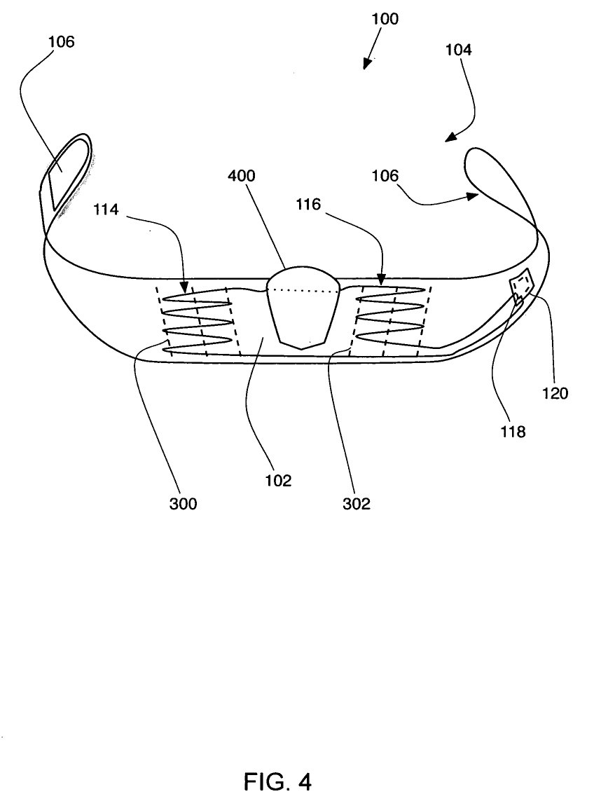

FIG. 4 depicts an embodiment of BPS 100 in which support pad 400 (corresponding to support pad 108 of FIG. 1) is a different shape, i.e., an elongated tapering semi-cylindrical form. The FIG. 4 embodiment includes the obliquely aligned strips 300, 302 of the FIG. 3 embodiment.

As an additional consideration, BPS 100 is producible in a variety of attractive colors, from bright and flashy pastels to more muted and practical solids. Additionally, a specially produced videotape or digital versatile disc (DVD) featuring exercises helping to relieve back pain is optionally offered bundled with each BPS 100. As a bundled package, the videotape or DVD may be positioned adjacent BPS 100, e.g., wrapped within the BPS, for sale as a unit.

FIG. 2 depicts another embodiment of BPS 100 making use of self-contained heating elements 200, 202 positioned on either side of lumbar support pad 108 and arranged so as to be adjacent a wearer's back muscles after a wearer has donned and positioned the BPS. As described, heating elements 200, 202 are self-contained and replaceable, e.g., fastener-attachable THERMACARE pads. Depending on the fastening mechanism used to affix heating elements 200, 202 to interior face 102 of BPS 100, the heating elements may be repositioned on the interior face to more readily, and anatomically, position the heating elements over particular targeted muscles. Further, repositioning of the heating elements 200, 202 may be necessitated due to physical differences between wearer's of BPS 100.

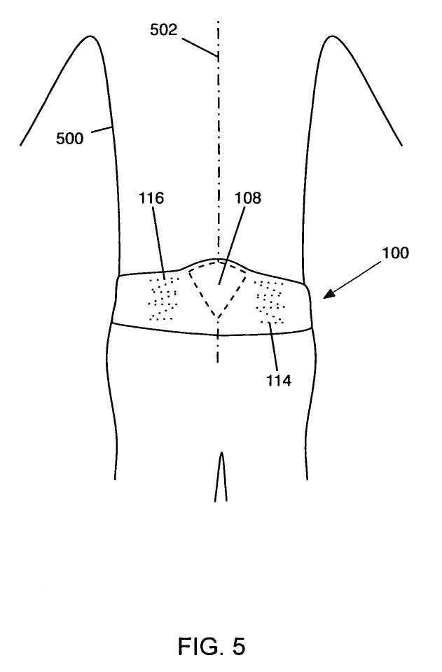

FIG. 5 depicts a rear view of BPS 100 as worn by a user 500. The spine of user 500 is aligned along central axis 502 (chain dashed line). As depicted, support pad 108 is positioned overlying the user's spine, generally along central axis 502 and heating elements 114, 116 are positioned adjacent to the support pad and positioned to apply heat to the user's back muscles.

Use of BPS 100 is simple and straightforward. First, the wearer simply wraps the unit around the wearer's body, positioning the belt so that lumbar support pad 108 lies flush against the lower back, just above the buttocks. For example, in use BPS 100 may be positioned adjacent the user's sacrolumbral region. After securely fitting the belt in a comfortable position on the wearer, the wearer then affixes the VELCRO strips together. Powering on the heating system (110, 112) of BPS 100 so that the heating system begins warming, the wearer is ready to engage in a desired activity. By way of example, a commuter may wish to don the BPS 100 while driving, ensuring a comfortable ride on a long drive to and from work. Similarly, a stay-at-home mother may choose to affix the unit to her body before embarking on a daily stroll through the park. While in use, the BPS 100 lumbar support pad 108 works to keep the wearer's spine properly aligned regardless of activity, while heating elements 110, 112 offer a soothing balm helping counteract spasming of muscles. In this manner, the wearer is able to sit in a seat and enjoy a comfortable and relaxing afternoon drive or complete a workout in a pain-free fashion. After use, the wearer removes BPS 100 from the wearer's body and folds and stores the system away until needed again.

BPS 100 affords consumers several significant benefits and advantages. Foremost, the ergonomically designed cushioning system, i.e., lumbar support pad 108, provides optimal comfort for lower back pain sufferers who find it difficult to complete exercise regimens or even to sit or recline for extended periods. Offering ample, heated support to the lower back, BPS 100 enables joggers, weightlifters, construction workers, office personnel, drivers, and even couch potatoes to stay comfortable and relaxed while engaging in these activities. As a result, painful sciatic episodes often occurring when a person is straining the back with strenuous endeavors or sitting upright for extended periods are effectively eliminated. Creatively configured, lumbar support pad 108 and connected warming system (heating elements 110, 112) help back pain sufferers sustain proper spinal alignment while also providing heat therapy to back muscles. As such, these aspects of BPS 100 are advantageous in preventing the onset of lower back pain in symptom-free users. Easy to use, the adjustable BPS belt permits users to don the unit in a matter of seconds. Manufactured of durable, high quality materials and components, BPS 100 is durable, e.g., withstands years of repeated use.

BPS 100 is a cleverly designed novel invention enabling consumers to exercise, work, or recline for extended periods in a relaxed and comfortable manner. Featuring an innovative combination of lumbar support and heat therapy configured into a wearable belt, the BPS provides sufferers of lower back pain with a simple and effective means of relief whenever needed. The BPS is usable by all who live with low back pain, as well as those who wish to prevent the onset of this condition.

It will be readily seen by one of ordinary skill in the art that the present invention fulfills many of the advantages set forth above. After reading the foregoing specification, one of ordinary skill will be able to affect various changes, substitutions of equivalents and various other aspects of the invention as broadly disclosed herein. It is therefore intended that the protection granted hereon be limited only by the definition contained in the appended claims and equivalents thereof.

What I claimed is:

1. A back protection system comprising:
a belt for placement on a user body;
a support pad operatively positioned at a central region of said belt;
a heating element positioned adjacent a side of said support pad and operatively connected with said belt; and
a power source connected to said heating element and arranged to cause said heating element to generate heat adjacent said support pad to a user.

2. A system as claimed in claim 1, wherein said support pad is positioned adjacent a user's back region when in use.

3. A system as claimed in claim 1, wherein said power source is reusable.

4. A system as claimed in claim 1, wherein said heating element is positioned solely on a side of said support pad in order to not heat said support pad.

5. A system as claimed in claim 1, wherein said support pad is alignable with a portion of a user spine.

6. A system as claimed in claim 1, wherein said belt includes a closure mechanism for positioning said belt on the user body.

7. A system as claimed in claim 6, wherein said closure mechanism is a hook and loop fastening system.

8. A system as claimed in claim 1, further comprising one or more reinforcement strips.

9. A system as claimed in claim 8, wherein said reinforcement strips are longitudinally aligned with the user body.

10. A system as claimed in claim 8, wherein said reinforcement strips are obliquely aligned with the user body.

11. A system as claimed in claim 1, wherein said heating element and said power source are an integrated unit.

12. A system as claimed in claim 1, further comprising a second heating element positioned adjacent a side of said support pad and operatively connected with said belt and said power source; and
wherein said power source is connected to said second heating element and arranged to cause said second heating element to generate heat adjacent said support pad to a user.

13. A system as claimed in claim 1, wherein said support pad is positioned adjacent a user's sacrolumbral region during use.

14. A system as claimed in claim 1, wherein said heating element is a chemically reactive heating element.

15. A system as claimed in claim 1, wherein said heating element is an electric-based heating element.

16. A method of applying heat to a user's back region using a back protection system having a support pad at a central region and at least one heating element adjacent a side of the support pad, comprising:
positioning the back protection system about a user's body;
aligning the support pad with the user's spine; and
activating the heating element to generate heat to the user's body.

17. A method as claimed in claim 16, wherein the generated heat is applied to the user's back region.

18. A method as claimed in claim 16, wherein the generated heat is applied in a region adjacent the user's spine.

19. A method as claimed in claim 16, further comprising:
performing an exercise after activation of the heating element.

20. A method as claimed in claim 16, wherein the generated heat warms the user's back muscles.
